(19) United States
(12) Patent Application Publication
(10) Pub. No.: US 2007/0088359 A1
(43) Pub. Date: Apr. 19, 2007

(54) Universal dynamic spine stabilization device and method of use

(76) Inventors: Richard W. Woods, Catonsville, MD (US); Hallet H. Mathews, Williamsburg, VA (US); Richard D. Guyer, Dallas, TX (US); Charles S. Theofilos, Palm Beach Gardens, FL (US); Gordon D. Donald, Fair Haven, NJ (US); Jean-Jacques Abitbol, San Diego, CA (US)

Correspondence Address: MANELLI DENISON & SELTER, 2000 M STREET NW SUITE 700, WASHINGTON, DC, 20036-3307, US

(21) Appl. No.: 11/348,468

(22) Filed: Feb. 7, 2006

Related U.S. Application Data
(60) Provisional application No. 60/649,965, filed on Feb. 7, 2005.

(51) Int. Cl. A61F 2/30; A61B 17/56

(52) U.S. Cl. 606/61

(57) ABSTRACT

Provided is a device, system, and method for the fixation of the spine, which provides dynamic support for spinal vertebra so as to better control load transfers and avoid deterioration of the bone of adjacent spinal vertebra. The present invention can be applied posteriorly to support the vertebra of the spine or can be adapted to attach to existing spinal constructs.

### BACKGROUND OF THE INVENTION

1. Field of the Invention

The present invention relates to devices, systems, and methods for the fixation of the spine. In particular, the present invention relates to a system that provides dynamic support for spinal vertebra so as to better control load transfers and avoid deterioration of the bone of adjacent spinal vertebra. More particularly, the present invention is directed to a device that can be used as part of a system applied posteriorly to support the vertebra of the spine or can be adapted to attach to existing spinal constructs to provide dynamic support of the spinal column so as to better manage load transfer and avoid deterioration of the bone of adjacent spinal vertebra.

2. Background of the Technology

Disease, the effects of aging, or physical trauma resulting in damage to the spine has been treated in many instances by fixation or stabilization of vertebra in spinal fusion procedures, which conventionally includes the use of polyaxial pedicle screws or hooks attached to longitudinally oriented spinal rods and firmly anchored in the bone of adjacent vertebrae. Such procedures and the associated fixation devices are well known and accepted throughout the medical community as being clinically successful. U.S. Pat. No. 4,648,388 issued to Steffee on Mar. 10, 1987, U.S. Pat. No. 5,129,900 issued to Asher et al. on Dec. 29, 1998, U.S. Pat. No. 5,102,412 issued to Roiozinski on Apr. 7, 1992, U.S. Pat. No. 6,613,050 issued to Wagner et al. on Sep. 2, 2003, and U.S. Pat. No. 6,964,665 issued to Thomas et al. on Nov. 15, 2005 are partially representative of the many various conventional devices employing spinal rods for the purpose of treating spinal problems.

While conventional procedures and devices have proven capable of providing reliable fixation of the spine, the resulting constructs typically provide a very high degree of rigidity to the operative levels of the spine. Unfortunately, this high degree of rigidity imparted to the spine by such devices can sometimes be excessive and result in the concentration of additional mechanical stresses on the vertebral levels adjacent to the levels that have undergone such fixation and fusion. Such stress can accelerate degeneration of the vertebra at these adjacent levels. The resulting degeneration can frequently require additional surgery; however, it is often very difficult to extend the earlier implanted construct to the next affected level. Further, even if such subsequent surgery can be successfully accomplished, the long term result is that the excessive rigidity and additional harmful stresses are merely transferred one more level in the spine.

There exists therefore a need to provide an improved device and system for fixation of levels of the spine. In particular there exists a requirement to provide a novel device and system for fixation of levels of the spine, which are adjacent to earlier fused levels and have, subsequent to the earlier surgery, suffered additional stress and degeneration by incorporating this novel device to the earlier implanted construct.

### SUMMARY OF THE INVENTION

The present invention meets the above identified need by providing a novel device and system for aligning and fixing vertebral bodies.

It is an object of the present invention to provide a posterior stabilization device which can be configured to provide an alternative to conventional rigid spinal fixation systems by stabilizing the affected vertebral segments while preserving an appropriate level of flexibility and protecting single or multiple adjacent levels from undue stress.

It is further an object of the present invention to provide a stabilization device that can be attached to an earlier implanted construct thereby providing stability to the adjacent vertebral level, which has suffered degeneration as a result of load transfer and undue stress from the rigid fixation of the earlier treated level.

It is further an object of the present invention to provide a stabilization device as described immediately above, wherein the device is designed and configured to attach to any of the conventional spinal fixation systems in use in a controlled, predictable, and measurable manner.

It is further an object of the present invention to provide a dynamic stabilization device wherein the degree of rigidity of the device can be adjusted intra-operatively.

It is further an object of the present invention to provide a hybrid spinal fixation device whereby one portion of the spinal rod can be relatively static or rigid while another portion of the spinal rod can be dynamic and capable of a degree of flexibility.

It is a further object of the present invention to provide a device having a dynamic rod component capable of absorbing mechanical shocks and thereby providing a protection against undue stress transfer to the vertebral body.

It is further an object of the present invention to provide a device that when connected to the vertebral column can function as a facet replacement in instances where the facets have been resected.

It is further an object of the present invention to provide a system for use in the fixation of vertebrae, wherein the system includes a dynamic rod component and attachment elements for connecting a first dynamic rod component to the vertebra as well as to static rod components or additional dynamic rod components as needed.

It is further an object of the present invention to provide a method for the fixation of adjacent vertebra whereby a degree of flexibility is maintained for the functional vertebra so as to avoid undue stress transfer and resulting degeneration of the vertebra.

### BRIEF DESCRIPTION OF THE DRAWINGS

The foregoing and other features of the present invention will become apparent to one skilled in the art to which the present invention relates upon consideration of the following description of the invention with reference to the accompanying drawings, wherein:

FIG's 1A-D respectively show top, front, side and oblique perspective views of a 1-level construct according to the present invention with bilateral dynamic spinal rods positioned in the bone attachment elements.

FIG's 2A-D respectively show top, front, side and oblique perspective views of a 1-level construct that has been augmented at an adjacent level with dynamic spinal rods according to the present invention, which are connected one to the other by a cross connector.

FIG's 3A-B respectively show top and oblique perspective views of the dynamic rod component of the present invention in a straight configuration attached by a connector block to a rigid one level construct.

FIG's 4A-B respectively show top and oblique perspective views of the dynamic rod component of the present invention in a curved configuration attached by a connector block to a rigid one level construct.

FIG's 5A-B respectively show top and oblique perspective views of the dynamic rod component of the present invention in a straight configuration attached by a universal cross connector to bilaterally disposed spinal rods of an existing construct.

FIG's 6A-D respectively show top, front, side and oblique perspective views of a dynamic spinal rod according to the present invention connected by a connector block to the terminal end of a spinal rod of a conventional or non-dynamic spinal rod.

FIG's 7A-D respectively show top, front, side and oblique perspective views of a 2-level construct according to the present invention with bilateral hybrid spinal rods.

FIG's 8A-D respectively show top, front, side and oblique perspective views of a 2-level construct according to the present invention with bilateral hybrid spinal rods, each having a cephalad disposed dynamic spinal rod component connected by a connector block to a caudad disposed rigid spinal rod component, the dynamic and rigid spinal rods being of different diameter.

FIG's 9A-D respectively show top, front, side and oblique perspective views of a 2-level construct according to the present invention with bilateral hybrid spinal rods having a cephalad disposed relatively dynamic portion and a caudad disposed relatively rigid portion.

FIG's 10A-B respectively show top and oblique views of a dynamic spinal rod in a medially arcing configuration according to the present invention connected to a universal cross connector.

FIG's 11A-B respectively show top and oblique views of a dynamic spinal rod in a straight configuration according to the present invention connected to a universal cross connector.

FIG's 12A-F show top views of examples of possible configurations of the dynamic spinal rod of the present invention, with FIGS. 12G and H showing examples of modular segments, which can be configured for selective insertion into the respective conformations of the dynamic spinal rods shown in FIG. 12D and FIG. 12E respectively, the purpose of the modular segments being to provide a selective degree of rigidity or flexibility of the respective dynamic spinal rod.

FIG's 13A-B respectively show oblique views of an exploded and an assembled modular dynamic rod component according to an alternative embodiment of the present invention.

FIG's 14A-C respectively show an alternative embodiment of the present invention including in FIG. 14A a perspective view of a flexible rod with a threaded portion, in FIG. 14B a perspective view of a threaded collar for engagement with the threaded portion of the flexible rod shown in FIG. 14A, and in FIG. 14C a cross-sectional view of the threaded collar of FIG. 14B.

FIG. 15A shows the threaded collar of FIG. 14B in partial connection with a clamping tool. FIG. 15B shows the threaded collar clamping tool for use in positioning the threaded collar of FIG. 14B onto the threaded portion of the flexible rod of FIG. 14A.

### DETAILED DESCRIPTION OF THE INVENTION

Detailed embodiments of the present invention are disclosed herein; however, it is understood that the following description and accompanying FIG's 1A-D to FIG's 15A-B are provided as being exemplary of the invention, which may be embodied in various forms without departing from the scope of the claimed invention. Thus, the specific structural and functional details provided in the description of present invention are non-limiting, but serve merely as a basis for the invention as defined by the claims provided herewith. The device described below can be modified as needed to conform to further development and improvement of materials without departing from the concept of the invention.

The present invention, as shown in FIG's 1A-D to FIG'S 15A-B provides a spine stabilization device generally shown at 10, the stabilization device 10, including a dynamic spinal rod component 12 having a rod first end 14 and a rod second end 16 with a relatively flexible portion 18 disposed between the rod first end 14 and a relatively rigid portion 20 disposed adjacent to the rod second end 16. With regard to the present invention, the terms rigid or rigidity refer to a greater degree of tensile strength of the rigid portion 20 as compared to the less rigid flexible portion 18 of the invention. The dynamic spinal rod 12, by convention, preferably has a generally circular shaped cross-section; however, alternative shapes, such as, for example oval, square, hexagonal, polygonal, elliptical, semi-circular, and substantially diametral with at least one flat side would be within the concept of the present invention.

The device 10 of the present invention, is capable of protecting the vertebra from load transfers and undue stress due to the provision of the flexible portion 18 of the dynamic spinal rod 12. The flexible portion 18 can be machined or otherwise formed by methods known in the art to measurably diminish the normal rigidity of the material in the section of the spinal rod 12 where the flexible portion 18 is desired. The length of the flexible portion 18 along the longitudinal axis of the spinal rod 12 as well as the depth or degree of machining or forming done to this section can be varied as necessary to increase or decrease the degree of flexibility imparted to the flexible portion 18. It is also within the concept of the present invention that the flexible portion 18 can be provided with greater flexibility over the relatively rigid portion 10 by altering the material composition during the manufacturing process in the section of the spinal rod where flexibility is desired.

The device 10 can be employed with additional components to form a spine stabilization system 22 that in addition to the dynamic spinal rod 12 can include at least one connector block 24, as best shown in FIG's 3A-B through FIG's 4A-B.

Additionally, the system 22 can include at least one cross connector 26 , as best shown in FIG's 2A-D and FIG's 5A-B. The connector block 24 and the cross connector 26 can be releasably secured to the dynamic spinal rod by set screws 28 configured to fit within designated set screw receiving portals 30. Alternative connection securing devices, such as, for example welding, threading, press or snap fittings, bayonet fittings, leur lock connections, and the like, as is known in the art, can also be employed without departing from the concept of the invention. It is also within the concept of the present invention to manufacture the connector block 24 or the cross connector 26 as an integral part with the dynamic spinal rod of the present invention. Additional connectors to bone, such as, for example hooks and plates, can also be used with the present invention.

As best shown in FIG's 12 A-F, the flexible portion 18 of the dynamic spinal rod 12 can be configured in a variety of shapes, all of which have the common characteristic of being configured to permit a greater capacity for bending or flexion of that flexible portion 18 as compared to the relatively rigid portion 20 of the spinal rod 12. Non-limiting examples of such flexible configurations include closed and opened spiral configurations as best shown in FIG's 12A-C, corrugated configurations as shown in FIG. 12D, or alternatively, unilateral or alternating bilateral ribbed configurations as shown in FIGS. 12E and 12F, respectively. As shown in FIGS. 12G and 12H, a modular segment 32 and 34, respectively can be provided as components to the system 22 for the purpose of selectively limiting the flexibility of the flexible portion 18 of the dynamic spinal rod 12. As shown in FIGS. 12G and 12H as they relate to respective FIGS. 12D and 12E, the modular segment 32, 34 can be selectively inserted into the corresponding conformation of the flexible portion 18 prior to the surgical procedure or, alternatively, can be inserted into the flexible portion 18 during the conduct of the surgical procedure, as decided by the surgeon. The inserted modular segments 12G and 12H can be removably or fixedly held in place in the respective flexible portion 18 by any method for connecting two elements including mechanical and chemical connections such as, for example, snap fitting, hooks, friction, welds, and adhesives.

As best shown in FIG's 7A-D, the present invention can be provided having a hybrid spinal rod, generally shown at 36. In this non-limiting example, each of the hybrid rods shown is made up of three components that include a cephalad disposed dynamic spinal rod component 12 connected by a connector block 24 to a caudad disposed rigid spinal rod component 38. In such a hybrid spinal rod the rigid spinal rod component 38 of the hybrid spinal rod 36 can be relatively static or rigid while the dynamic spinal rod component 12 of the hybrid spinal rod 36 can be dynamic and capable of a degree of flexibility. The third connector block 24 component provides a releasably secure connection between the other two components. It is also within the concept of the present invention to reverse the disposition of the dynamic spinal rod 12 component and the rigid spinal rod 38 component on bilateral hybrid spinal rod 36, thus allowing maximum selectivity for the position of the flexible portion.

As best shown in FIGS. 2A-D, 3A-B, 4A-B, and 5A-B, when in use the system of the present invention provides the necessary flexibility to provide support to a deteriorating portion of the spine and to permit the attachment of the dynamic spinal rod 12 to an existing spinal construct 40, without the necessity to remove or replace any of the earlier implanted devices. The present device is configured to be adaptable for attachment to any other construct currently in use. In such an application, the measure and controlled flexibility in the system 22 of the present invention allows the new construct to contour around the existing spinal constructs 40 and attach to adjacent levels without the need for intra-operative bending of the dynamic spinal rod. This capability of the present invention provides a device 10 and procedure whereby a surgeon can stabilize a vertebral level that has begun to deteriorate or is in danger of deterioration as a result of load transfers and undue stress on that level from an overly rigid existing construct 40 at an adjacent level.

In an alternative embodiment of the present invention, the device, as best shown in FIG's 14A-15A can include an alternative flexible rod 42 being flexible along an intermediately disposed portion 44 capable of receiving a collar 46 that is sized and configured to fit circumferentially around the alternative flexible rod 42.

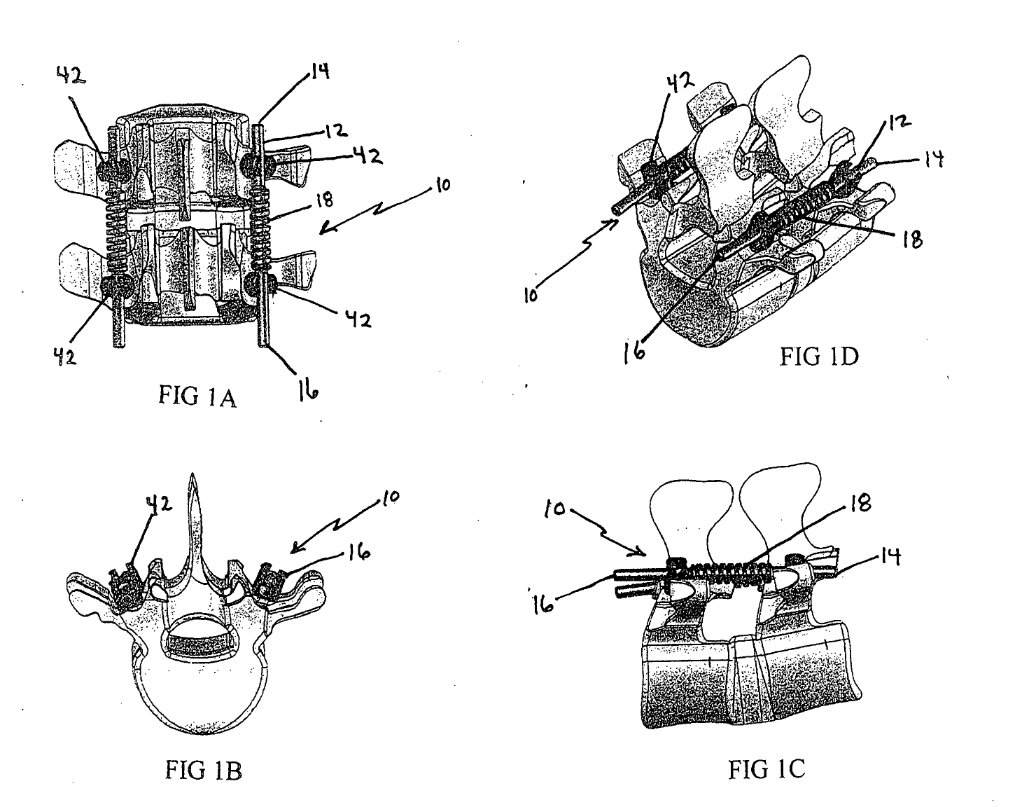

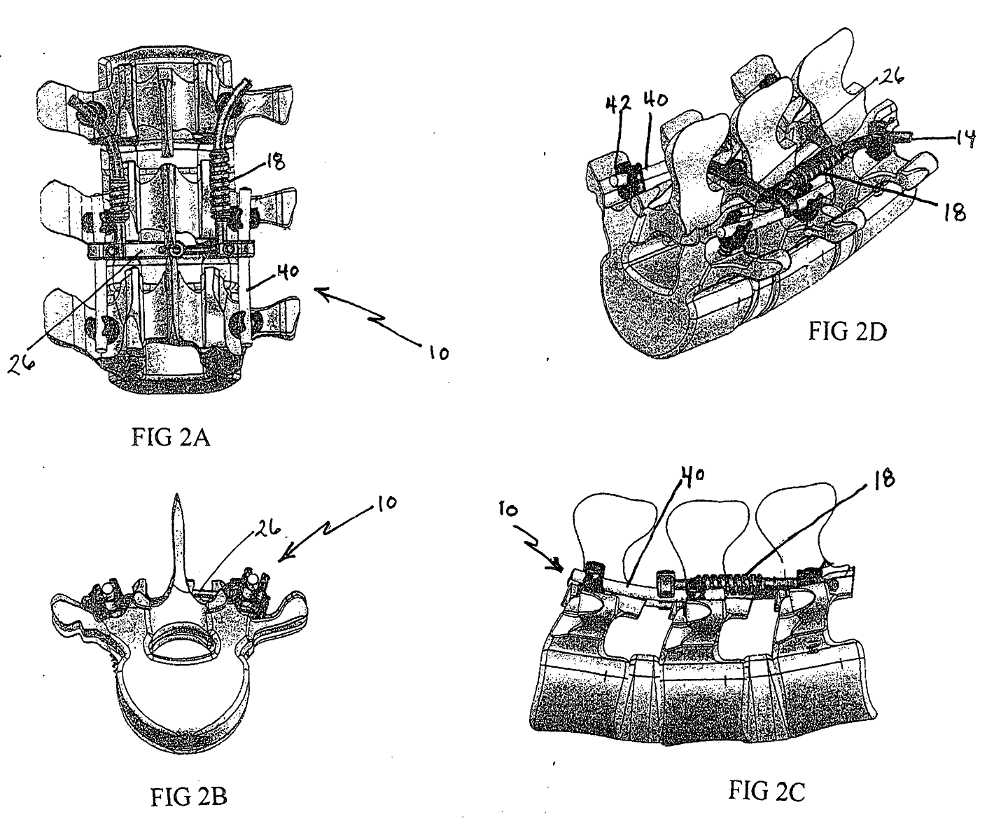

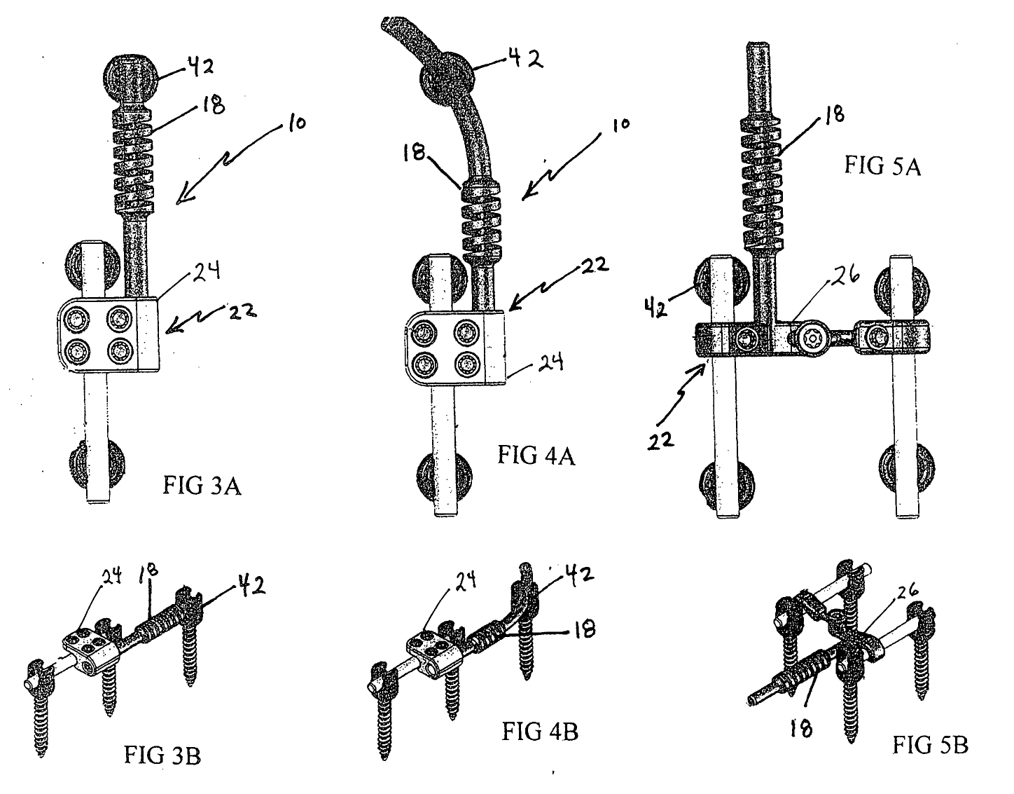

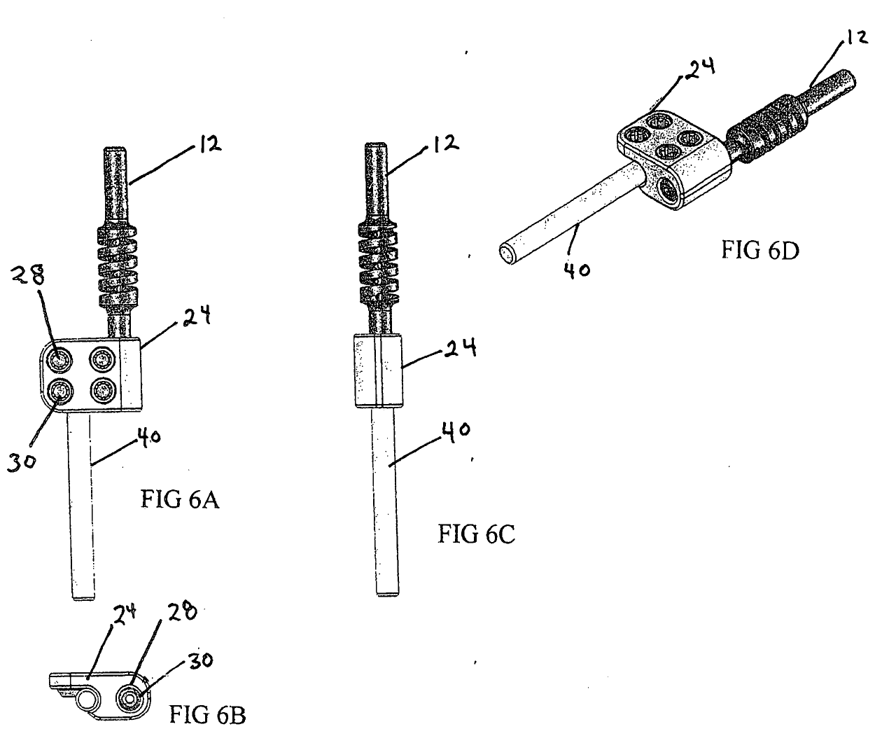

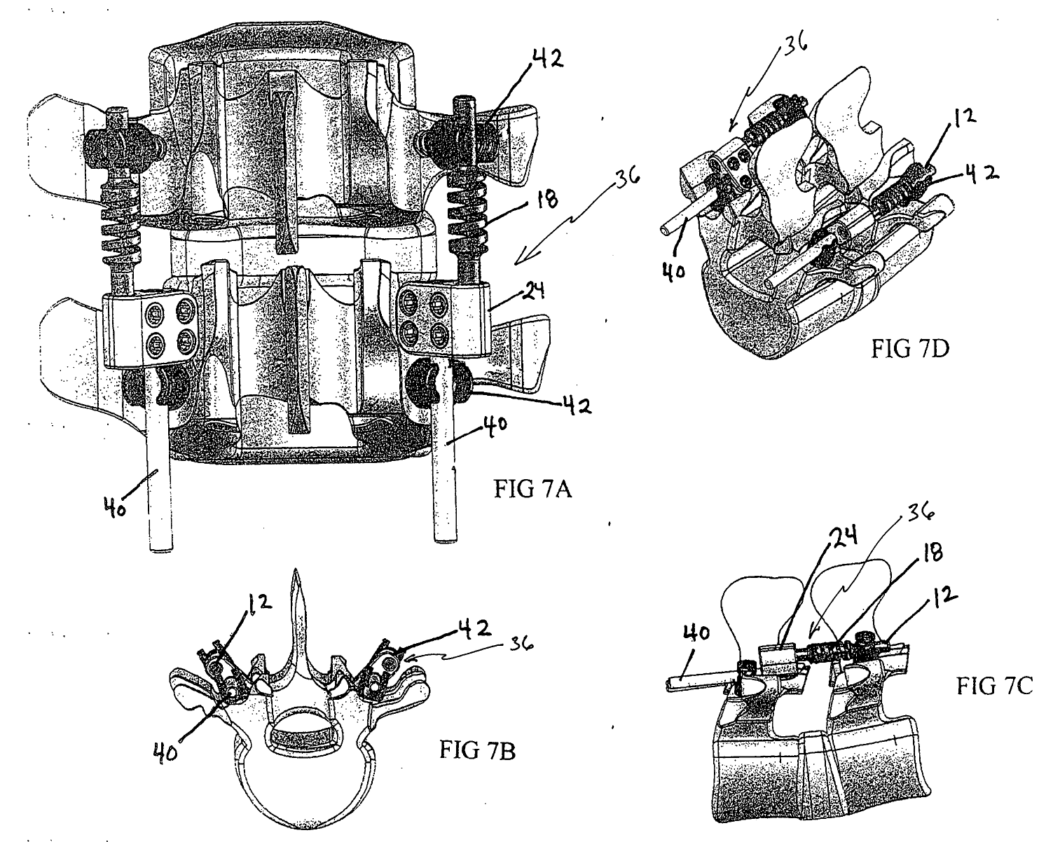

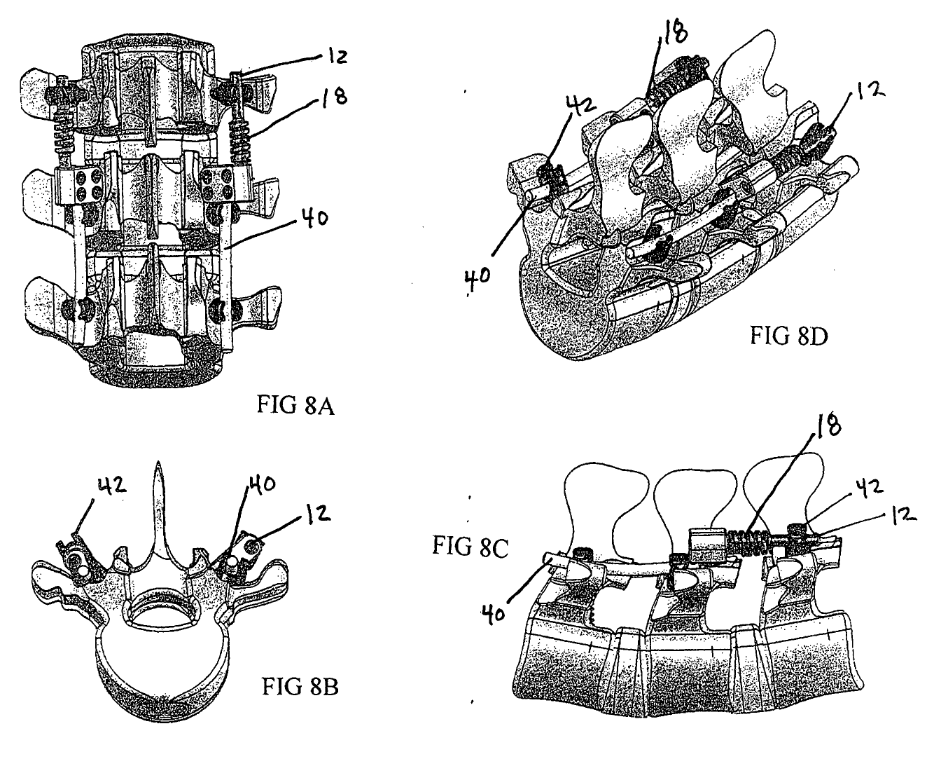

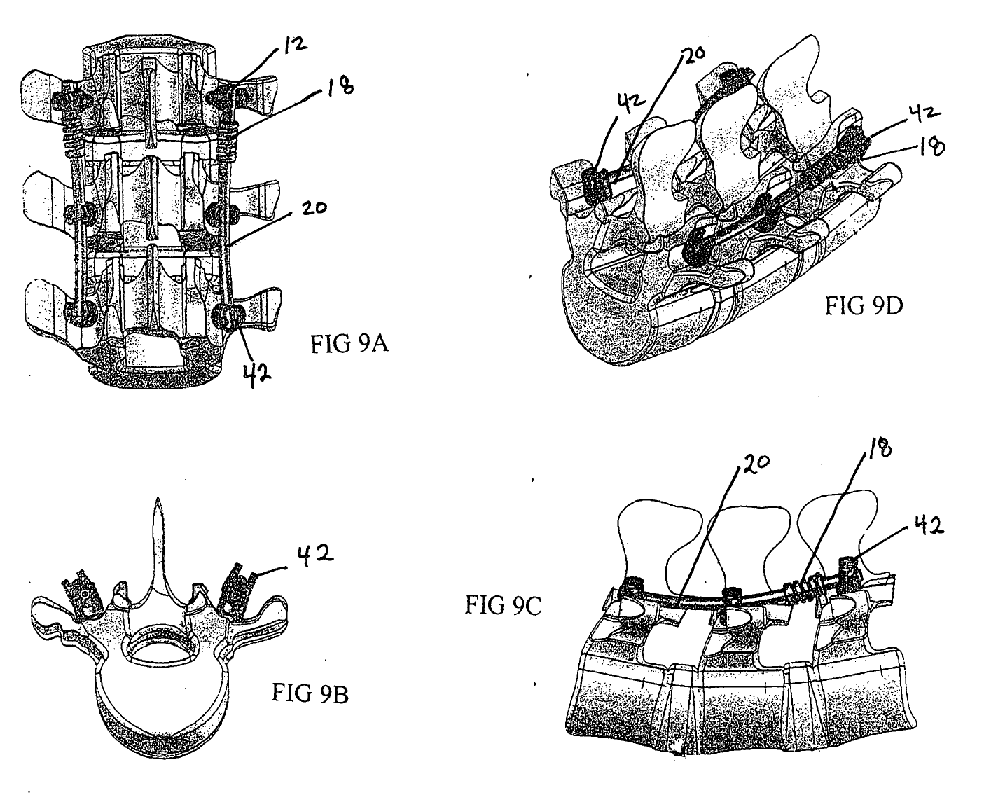

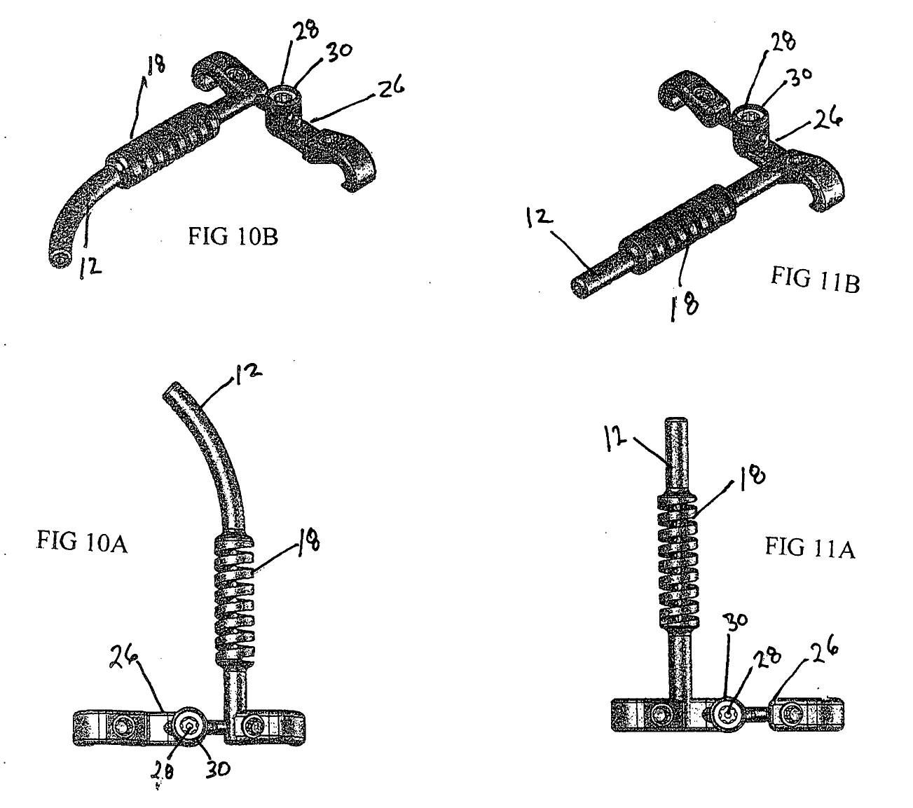

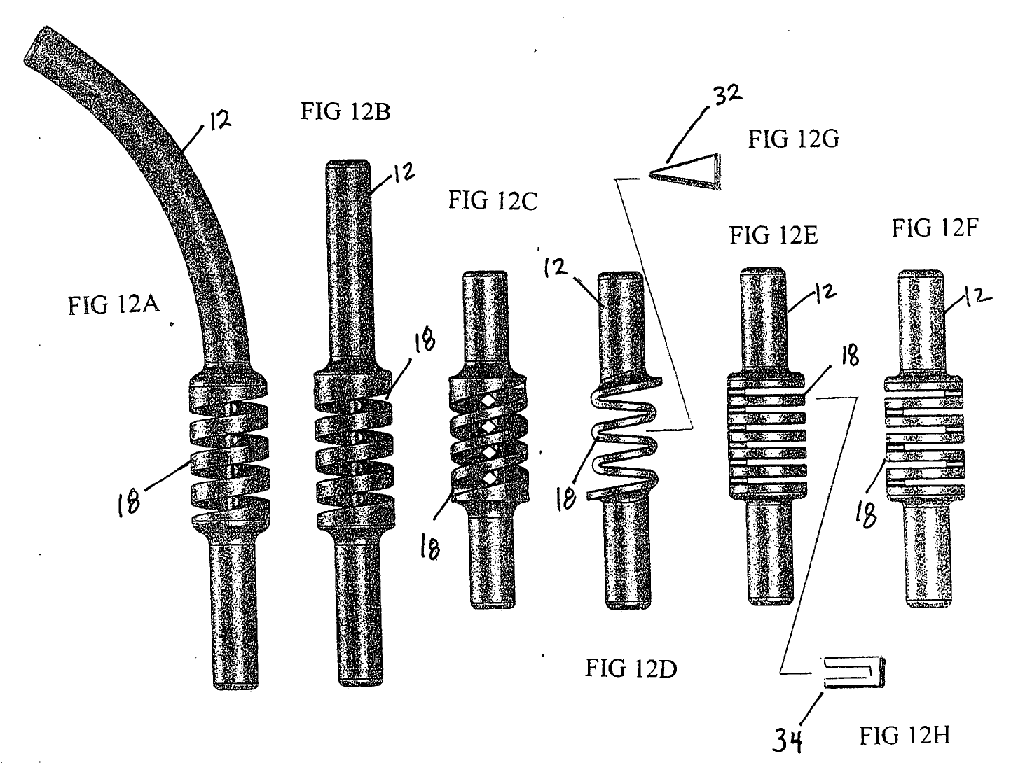

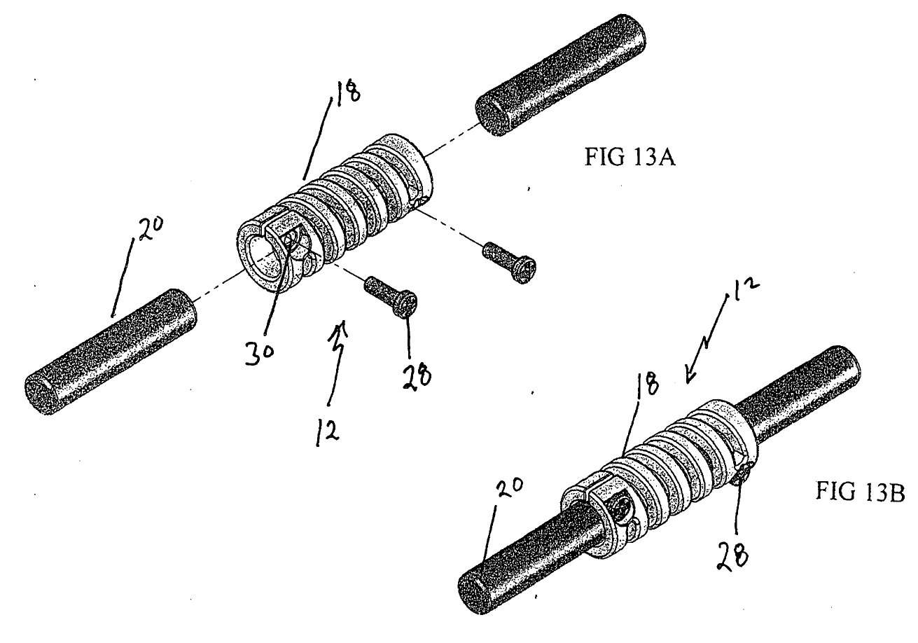

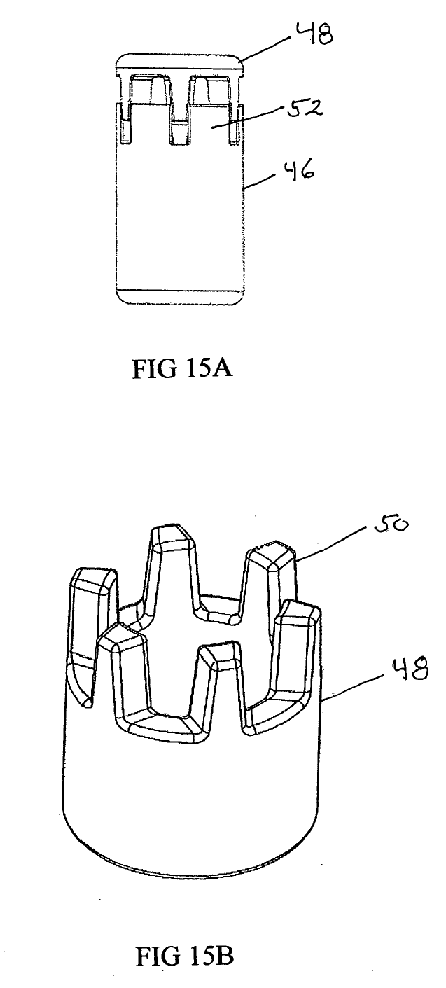

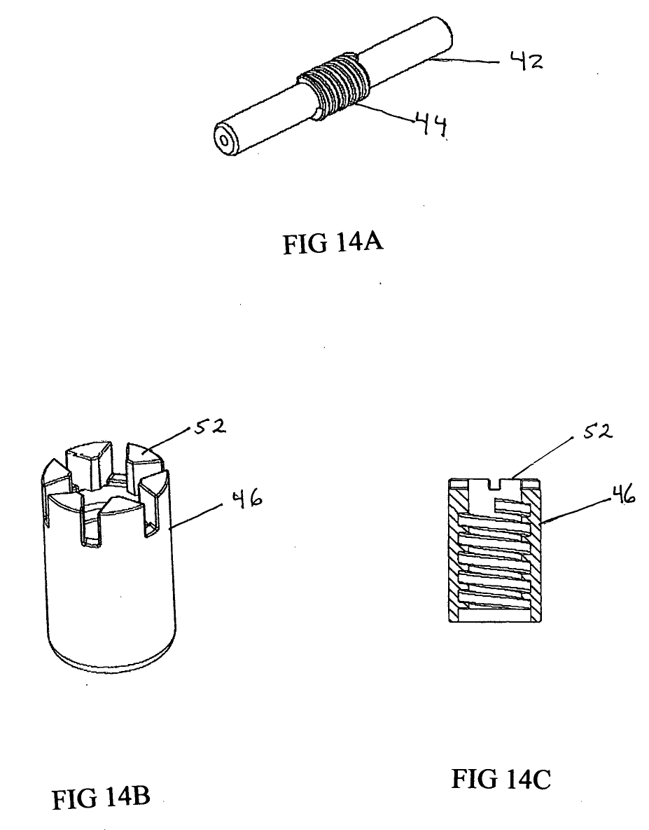

Preferably, the intermediately disposed portion 44 is threaded and the receiving collar 46 is a threaded collar having a complimentary thread to the intermediately disposed portion 44. The collar 46 is relatively rigid as compared to the flexible rod 42 and when positioned over the intermediately disposed portion 44, imparts a relatively high degree of rigidity to that portion of the flexible rod 42 covered by the collar 46. FIG. 1 5A shows collar 46 in partial connection with a clamping tool 48. The clamping tool 48, as best shown in FIG. 15B is configured to effectively spread the locking members 52 of the collar 46 so as to facilitate the passage of the collar 46 circumferentially over the length of the alternative flexible rod 42. By use of the clamping tool 48, the collar 46 can be selectively position along the length of the alternative flexible rod 42. The resilient quality of the material of the collar 46 is such that upon removal of the spreading members 50 of the clamping tool 48 from engagement with the locking members 52 of the collar 46, the locking members will return to a inwardly directed configuration and a locking contact with the alternative flexible rod 42. By use of this alternative embodiment of the present invention, flexible rod construct can be provided that permits the user to restrict or inhibit the flexibility of the rod at any intermediate position desired along the length of the rod.

The dynamic spinal rod 12 and the alternative flexible rod 42 can be manufactured as an integral component by methods known in the art, to include, for example molding, casting, forming or extruding, and machining processes. Alternatively, as shown in FIG's 13A-B, the dynamic spinal rod 12 can be manufactured as a modular component that allows for custom assembly to fit the needs of a particular subject. The dynamic spinal rod 12 and the alternative flexible rod 42 can be manufactured in any length or size desired; however it is contemplated that a rod diameter of 3 mm to 7 mm is preferred at the rigid portion or in the case of the alternative flexible rod 42 at the non-threaded portion of the rod.

The components of the present invention can be manufactured using methods and materials as known in the art such as for example, implant grade metallic materials, such as titanium, cobalt chromium alloys, stainless steel, and the like. It is also within the concept of the present invention that the components can be manufactured from any bio-compatible materials such as composite materials or plastics. Non-limiting examples of such materials include polyetheretherketone (PEEK) or polyaryletherketone (PAEK), or composites thereof, which can incorporate carbon fibers or similar materials. The materials used in the manufacture of the device and components of a kit can be radiopaque or radiolucent.

It is also within the concept of the present invention to provide a kit, which includes the device of the present invention as well as other components discussed above. Such a kit can include various lengths, diameters, connector blocks, cross connectors, bone connector elements, such as for example pedicle screws and the like, and different embodiments of the above described components. Instructions and packaging materials can also be included in such a kit. Such a kit can be provided in sterile packaging for opening and immediate use in the operating room.

The method of the present invention provides for the determination that a subject is in need of or potentially in need of a procedure to implant the device of the present invention, the operable implanting of the device and, if necessary, the connection of the device to any existing construct on an adjacent level. Operative techniques and tools for implantation of the device can be employed as necessary in accordance with safe surgical practices.

Each of the embodiments described above are provided for illustrative purposes only and it is within the concept of the present invention to include modifications and varying configurations without departing from the scope of the invention that is limited only by the claims included herewith.

What is claimed is:

1. A spinal stabilization device, comprising:
an elongated dynamic spinal rod, having a first end and a second end, said first end and said second end being sized and configured to be secured at said first and second ends to at least one spinal implant; and
a flexible portion and at least one rigid portion, said flexible portion being positioned between said first end and said second end, said flexible portion having a predetermined degree of flexibility.

2. The device of claim 1, wherein said dynamic spinal rod is between approximately 1 mm and 10 mm cross-sectional diameter at said at least one rigid portion.

3. The device of claim 1, wherein said dynamic spinal rod is between approximately 3 mm and 7 mm cross-sectional diameter at said at least one rigid portion.

4. The device of claim 1, wherein said flexible portion is configured to undermine the normal rigidity of the material so as to impart a capacity to have a selected degree of flexibility under the normal stress for a vertebral column.

5. The device of claim 4, wherein said configuration of the flexible portion is a spiral configuration.

6. The device of claim 4, wherein said configuration is a closed spiral configuration.

7. The device of claim 4, wherein said configuration is an open spiral configuration.

8. The device of claim 4, wherein said configuration is a ribbed or corrugated configuration.

9. The device of claim 8, wherein said configuration is a unilaterally directed ribbed configuration.

10. The device of claim 8, wherein said configuration is an alternating directed ribbed configuration.

11. The device of claim 1, further comprising at least one modular segment sized and configured to complement and attach to the configuration of said flexible portion, wherein said modular segment measurably restricts the flexibility of said flexible portion.

12. The device of claim 1, wherein said at least one spinal implant is selected from the group consisting of a pedicle screw, a rod connector block, a cross connector, and a bone connector element.

13. A spinal stabilization device, comprising:
an elongated dynamic spinal rod, the length of said spinal rod terminating in a first end and a second end, said first end and said second end being sized and configured to be secured at said first and second ends to at least one spinal implant, said spinal rod being flexible;
a rigid collar sized and configured to fit circumferentially around said spinal rod and to cover at least a portion of the length of said spinal rod, said rigid collar, when circumferentially disposed around said spinal rod inhibits flexibility of the underlying portion of said spinal rod; said rigid collar having two ends, at least one of said two ends being configured to form a clamping component, said clamping component when in a clamped position locks said rigid collar in a relative position to said spinal rod.

14. The device of claim 13, further comprising a clamping tool sized and configured to releasably engage said clamping component and to slidably traverse circumferentially said spinal rod to selectively position said rigid collar on said spinal rod, wherein said clamping tool is configured to hold said clamping component of said rigid collar in an unclamped position until said clamping tool is disengaged from said clamping component.

15. The device of claim 13, wherein said dynamic spinal rod is between approximately 1 mm and 10 mm cross-sectional diameter at said at least one rigid portion.

16. The device of claim 13, wherein said dynamic spinal rod is between approximately 3 mm and 7 mm cross-sectional diameter at said at least one rigid portion.

17. The device of claim 13, wherein said spinal rod and said rigid collar are threadably engaged.

18. The device of claim 13, wherein said clamping component is releasably secured to said clamping component receptor portion of said spinal rod.

19. The spinal rod of claim 13, wherein said clamping component is fixedly secured to said clamping component receptor portion of said spinal rod.

20. The device of claim 13, wherein said at least one spinal implant is selected from the group consisting of a pedicle screw, a rod connector block, a cross connector, and a bone connector element.

21. A system for stabilizing the spine of a subject, comprising:
an elongated dynamic spinal rod, having a first end and a second end, said first end and said second end being sized and configured to be secured within a spinal rod receiving member;
a flexible portion positioned between said first end and said second end, said flexible portion having a selectable degree of flexibility; and
at least one connector device sized and configured to connect said elongated dynamic spinal rod to a spinal rod of an existing spinal construct in said subject.

22. The system of claim 21, wherein said at least one connector is a connector block.

23. The system of claim 21, wherein said at least one connector is a cross connector.

24. The system of claim 21, wherein said dynamic spinal rod is between approximately 1 mm and 10 mm cross-sectional diameter at said at least one rigid portion.

25. The system of claim 21, wherein said dynamic spinal rod is between approximately 3 mm and 7 mm cross-sectional diameter.

26. The system of claim 21, wherein said flexible portion is configured to undermine the normal rigidity of the material so as to impart a capacity to have a selected degree of flexible under the normal stress for a vertebral column.

27. The system of claim 26, wherein said configuration of the flexible portion is a spiral configuration.

28. The system of claim 26, wherein said configuration is a ribbed or corrugated configuration.

29. The system of claim 21, further comprising at least one modular segment sized and configured to complement and attach to the configuration of said flexible portion, wherein said modular segment measurably restricts the flexibility of said flexible portion.

30. The system of claim 21, wherein said dynamic spinal rod further comprises a flexible portion and a rigid portion.

31. A method of fixation of the spine of a subject, comprising:
determining that a subject is in need of or potentially in need of a procedure to implant the device of claim 1; and
connecting the device to any existing construct on an adjacent level.

32. A kit for use in a surgical procedure, said kit comprising:
at least one device according to claim 1;
at least one additional component selected from the group consisting of a connector block, a cross connector, and a device installation instrument;
a sealable packaging container for said kit.
